(19) United States
(12) Patent Application Publication
(10) Pub. No.: US 2020/0345876 A1
(43) Pub. Date: Nov. 5, 2020

(54) Sterilizer

(71) Applicant: Share Light Co., LTD., Gyeonggi-do (KR)

(72) Inventor: Eun Hyun PARK, Gyeonggi-do (KR)

(73) Assignee: Share Light Co., LTD.

(21) Appl. No.: 16/859,489

(22) Filed: Apr. 27, 2020

(30) Foreign Application Priority Data
Apr. 30, 2019 (KR) 10-2019-0050481
Jul. 24, 2019 (KR) 10-2019-0089647

(51) Int. Cl. A61L 2/10; H02S 40/30; A61L 2/26

(52) U.S. Cl. CPC A61L 2/10; H02S 99/00; A61L 2/26; H02S 40/30

(57) ABSTRACT

Disclosed is a sterilizer for sterilizing a container with ultraviolet rays. The sterilizer includes a light-emitting element including at least one semiconductor light-emitting diode that emits ultraviolet rays directed to the container; a power supply for supplying electrical energy to the light-emitting element; a casing having a main body in which the power supply is provided, and a neck portion lying under the main body, the neck portion being configured to join to the container; and a circuit element for detecting a change in electrical energy of the power supply to control the light-emitting element.

### CROSS REFERENCE TO RELATED APPLICATION

This application is claims the benefit and priority of Korean Patent Application No. 10-2019-0050481, filed on Apr. 30, 2019 and Korean Patent Application No. 10-2019-0089647, filed on Jul. 24, 2019. The entire disclosures of the applications identified in this paragraph are incorporated herein by references.

### FIELD

The present disclosure generally relates to a sterilizer and, in particular, to a sterilizer featuring easy installation for use.

### BACKGROUND

This section provides background information related to the present disclosure which is not necessarily prior art.

FIG. 1 schematically illustrates an example of a sterilization container described in Korean Patent Publication No. 10-1875681, where the sterilization container comprises a body 10, a closure 20, a power supply 30, an ultraviolet lamp 40, a packing element 50, and an indicator 60.

The closure 20 is arranged on the upper portion of the body 10, and the power supply 30 is arranged on the lower portion of the body 10. The power supply 30 provides power to the ultraviolet lamp 40. The ultraviolet lamp 40 is provided inside the body 10 and sterilizes a liquid contained in the body 10. The packing element 50 is arranged between the power supply 30 and the ultraviolet lamp 40 and configured to prevent any extraneous matter in the body 10 from entering the power supply 30. The indicator 60 is provided outside the body 10.

FIG. 2 schematically illustrates an example of a UV sterilization overcap assembly for a feeding bottle nipple described in Korean Utility Model Publication No. 20-0380993. The reference numerals have been changed for convenience of explanation herein.

The UV sterilization overcap assembly 2 for a feeding bottle nipple includes a cap 7 on top of an electronic circuit board, a solar cell (also referred to as a photovoltaic cell) 5, a rechargeable battery 8, a power switch 6, an electronic circuit board 9, and an ultraviolet lamp 4. The cap 7 is placed over the electronic circuit board 9, and the solar cell 5 and the power switch 6 are arranged on the cap 7. In addition, the rechargeable battery 8 is also located on the electronic circuit board 9. The ultraviolet lamp 4 is arranged between the overcap assembly 2 for a feeding bottle nipple and the cap 7 on top of the electronic circuit board.

The ultraviolet lamp 40 in FIG. 1 is provided at the bottom of the container. The packing element 50, which is arranged between the power supply 30 and the ultraviolet lamp 40, ensures that no extraneous matter enters the power supply 30. However, the packing element 50 deteriorates and becomes loosened with time after repeated use, possibly causing the extraneous matter to get into the power supply 30.

The UV sterilization overcap assembly 2 for a feeding bottle nipple in FIG. 2 is provided over the feeding bottle 3 and the nipple 1. This UV sterilization overcap assembly 2 is configured to sterilize the feeding bottle 3 as well as the nipple 1, but not the liquid contained in the bottle. Further, since the electronic circuit board 9 is not waterproof, it has to be disassembled for cleaning.

### SUMMARY

This section provides a general summary of the disclosure and is not a comprehensive disclosure of its full scope or all of its features.

According to one aspect of the present disclosure, there is provided a sterilizer for sterilizing a container with ultraviolet rays, the sterilizer including: a light-emitting element including at least one semiconductor light-emitting diode that emits ultraviolet rays directed to the container; a power supply for supplying electrical energy to the light-emitting element; a casing having a main body in which the power supply is provided, and a neck portion lying under the main body, the neck portion being configured to join to the container; and a circuit element for detecting a change in electrical energy of the power supply to control the light-emitting element.

According to another aspect of the present disclosure, there is provided a sterilizer for sterilizing a container with ultraviolet rays, the sterilizer including: a light-emitting element including at least one semiconductor light-emitting diode that emits ultraviolet rays directed to the container; a solar cell for supplying electrical energy to the light-emitting element; and a casing having a main body in which the solar cell is provided, and a neck portion extending downward on opposite sides of the solar cell, the neck portion having an outer surface and an inner surface, wherein the container joins to the outer surface and the inner surface of the neck portion.

According to another aspect of the present disclosure, there is provided a sterilizer coupled to a container, the sterilizer including: a casing including a coupling portion to join to the container; a light-emitting element for emitting light directed to the interior of the container, the light-emitting element including a first semiconductor light-emitting diode for emitting ultraviolet rays; and a solar cell provided in the casing, the solar cell being electrically connected with the light-emitting element to supply electrical energy to the light-emitting element.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 schematically illustrates an example of a sterilization container described in Korean Patent Publication No. 10-1875681.

FIG. 2 schematically illustrates an example of a UV sterilization overcap assembly for a feeding bottle nipple described in Korean Utility Model Publication No. 20-0380993.

FIG. 3 illustrates an exemplary embodiment of a sterilizer according to the present disclosure.

FIG. 4 illustrates another exemplary embodiment of a sterilizer according to the present disclosure.

FIG. 5 illustrates another exemplary embodiment of a sterilizer according to the present disclosure.

FIG. 6 illustrates a method of operating a sterilizer according to the present disclosure.

FIG. 7 is a flow chart describing the operations of a sterilizer according to the present disclosure.

FIG. 8 is a schematic block diagram showing a power supply, a circuit element, and a light-emitting element according to the present disclosure.

FIG. 9 illustrates another exemplary embodiment of a sterilizer according to the present disclosure.

FIG. 10 is a conceptual view of a sterilizer according to the present disclosure.

FIGS. 11 and 12 illustrate an exemplary embodiment of a sterilizer according to the present disclosure.

FIG. 13 illustrates another exemplary embodiment of a sterilizer according to the present disclosure.

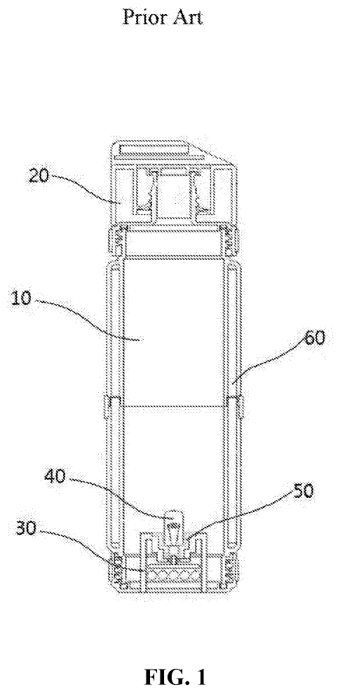

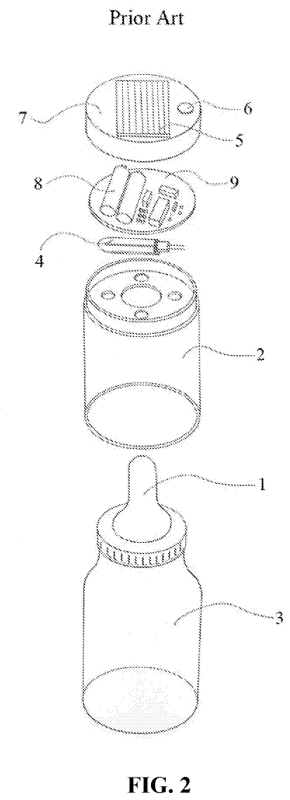

### DETAILED DESCRIPTION

The present disclosure will now be described in detail with reference to the accompanying drawing(s).

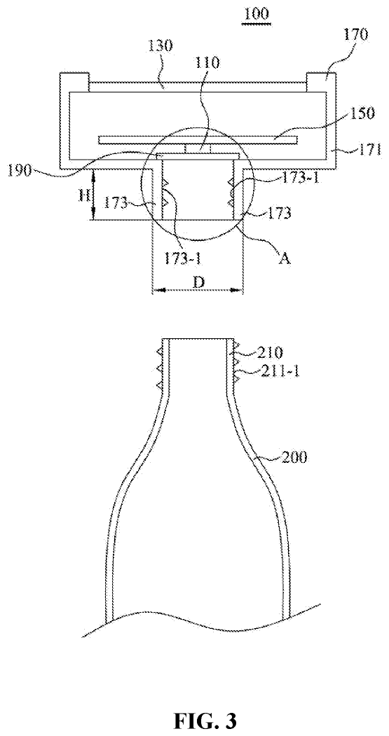

FIG. 3 illustrates an exemplary embodiment of a sterilizer 100 according to the present disclosure.

The sterilizer 100 includes a light-emitting element 110, a power supply 130, a circuit element 150, and a casing 170. The sterilizer 100 may be used as a lid or cap positioned at an opening (or inlet) 210 of a container 200 where an intended liquid is contained, such that the sterilizer 100 can sterilize both the liquid and the container 200. As an example, the container 200 may be any type of a small bottle, a recyclable water bottle, or a beverage bottle. The opening of the container 200 is designed to have a standard size.

The light-emitting element 110 may include at least one semiconductor light-emitting diode. An example of the semiconductor light-emitting diode is an ultraviolet LED. The light-emitting element 110 can emit ultraviolet rays, such as UVC, for example. Moreover, the light-emitting element 110 may further include another semiconductor light-emitting diode, which can be an LED that emits visible light. With ultraviolet rays being invisible, it may be difficult to see whether the ultraviolet LED is ON/OFF, especially if the light-emitting element 110 only has an ultraviolet LED. As such, the light-emitting element 110 preferably has an ultraviolet LED as well as a visible LED.

The power supply 130 provides electrical energy (e.g., current and/or voltage) to the light-emitting element 110. The power supply 130 includes a solar cell. Typically, a solar cell is a device that generates electrical energy or electricity using the energy of light, which enables the power supply 130 to provide electrical energy to the light-emitting element 110. In addition, the power supply 130 may further include a battery that can store electrical energy generated by the solar cell. Therefore, the sterilizer 100 can utilize the electrical energy stored in the battery, even when the sun is not available at the time of need.

The circuit element 150 is electrically connected to the power supply 130 and the light-emitting element 110 and controls turning on/off the light-emitting element 110. The circuit element 150 allows the electrical energy from the power supply 130 to be provided to the light-emitting element 110 such that the light-emitting element 110 would be turned on. Also, the circuit element 150 cuts off the electrical energy supplied from the power supply 130 to the light-emitting element 110 such that the light-emitting element 110 would be turned off.

The casing 170 encloses the power supply 130, the light-emitting element 110, and the circuit element 150. The casing 170 includes a main body 171 and a neck portion 173.

The main body 171 may be a portion of the sterilizer 100 that the user would touch when he tries to put the container 200 and the sterilizer 100 together. It is the main body 171 in which the light-emitting element 110, the power supply 130, and the circuit element 150 are arranged.

The neck portion 173 lies under the main body 171 and is configured to join to the container 200, surrounding it. Alternatively, if ultraviolet rays are not emitted to the outside, the neck portion 173 may be fitted into the container 200.

The neck portion 173 has a width smaller than the width of the main body 171.

For example, the container 200 has an opening 210, and an outer surface 211-1 of the opening 210 has protrusions which are then meshed with its corresponding grooves formed on an inner surface 173-1 of the neck portion 173 to have matching finishes. Additionally, or alternatively, the projection part of the neck portion 173 and the projection part of the opening 210 may be engaged together in a rotatable manner. As an example, the container 200 may be a PET plastic bottle.

The power supply 130 may be arranged inside the casing 170 and above the casing 170. The power supply 130 may lie over the entire casing 170.

The light-emitting element 110 emits ultraviolet rays downstream of the casing 170, such that the container 200 located below the casing 170 as well as a material contained in the container 200 can be sterilized by the ultraviolet rays from the light-emitting element 110.

A light-transmitting element 190 can be provided between the light-emitting element 110 and the neck portion 173. The light-transmitting element 190 passes ultraviolet rays, and may be, for example, sapphire, quartz, or the like. Also, the light-transmitting element 190 can be adapted to prevent an extraneous matter from entering the casing 170.

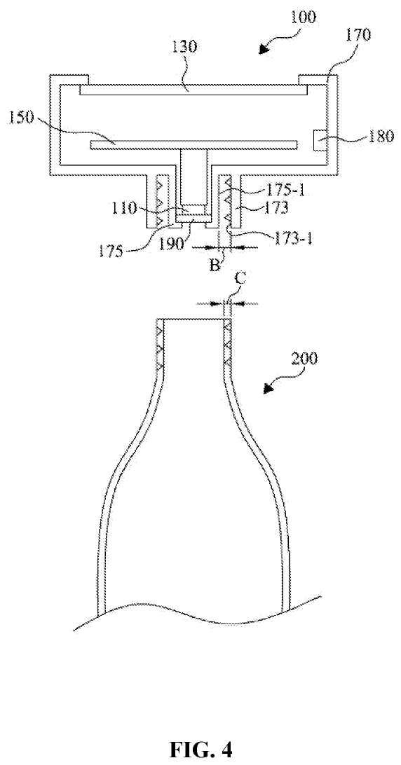

FIG. 4 illustrates another exemplary embodiment of a sterilizer 110 according to the present disclosure.

A light-emitting element 110 is provided in a neck portion 173, and protrusions 175 may be formed within the neck portion 173, surrounding the light-emitting element 110.

A circuit element 150 is partially elevated such that the light-emitting part 110 may be placed on the protrusions 175. Since ultraviolet rays from the light-emitting element 110 enters the inside of the container 200 and emits light, ultraviolet rays can reach far back into the container 200.

There is a gap B between an inner surface 173-1 of the neck portion 173 and an outer surface 175-1 of the protrusion 175. The gap B is approximately equal to the thickness C of the container 200. In addition, the sterilizer 100 may include an indicator 180. The indicator 180 can inform the user as to how the operation of the light-emitting element 110 has been terminated. The indicator 180 is electrically connected to the circuit element 150. Examples of the indicator 180 may include a speaker, an LED, a display, or the like. The indicator 180 is preferably an LED, which is more energy efficient.

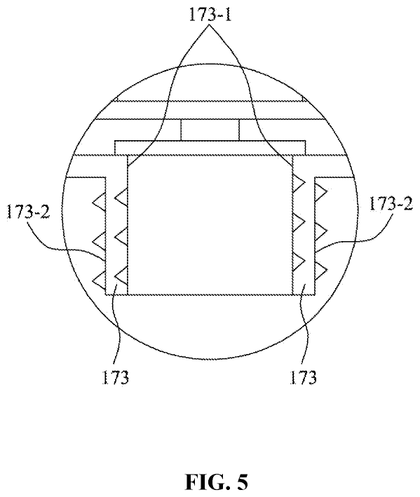

FIG. 5 illustrates another exemplary embodiment of a sterilizer according to the present disclosure.

In FIG. 5, another example of the neck portion 173 of FIG. 3 is provided in an enlarged view.

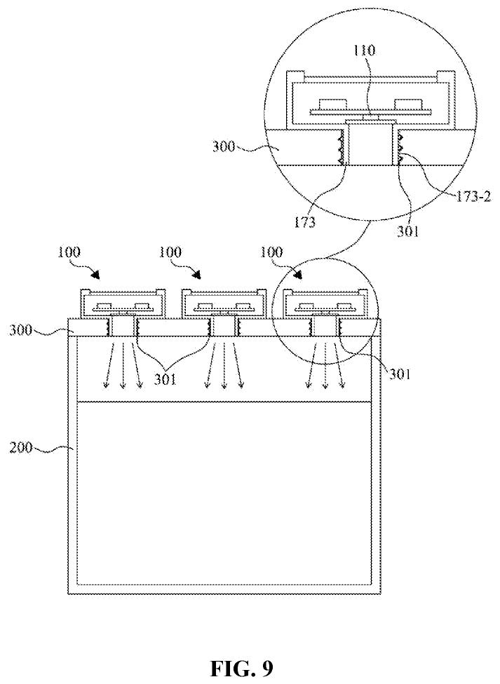

The neck portion 173 has an outer surface 173-2 on which grooves or protrusions may be formed, and optionally, it may further include a packing element (not shown). These grooves, protrusions, and packing elements allow the sterilizer 100 (FIG. 3) to be secured to the container 200 (FIG. 3) or to a lid 300 (FIG. 9) of the container 200. The outer surface 173-2 of the neck portion 173. The configuration of the outer surface 173-2 of the neck portion 173 discussed here may ensure that the sterilizer 100 is firmly secured to the container 200 or to the lid 300 of the container 200, while increasing the sealing force between the sterilizer 100 and the container 200 or the lid 300 of the container 200.

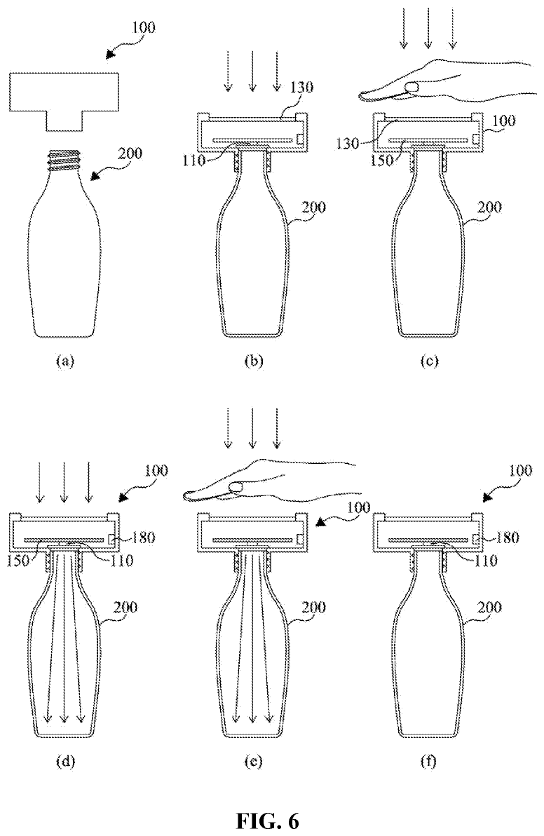

FIG. 6 illustrates a method of operating a sterilizer according to the present disclosure.

To begin with, the sterilizer 100 and the container 200 are prepared, as illustrated in FIG. 6A.

Referring next to FIG. 6B, a solar cell of the power supply 130 converts the energy of light into electrical energy. The light-emitting element 110 will likely have a reduced lifespan if the light-emitting element 100 is configured to turn on whenever electrical energy is generated. Therefore, it is desirable that the light-emitting element 110 is configured to turn on when and only when the user wants a sterilization process to be done, regardless of the generation of electrical energy.

The light directed toward the solar cell is then temporarily blocked as shown in FIG. 6C, and the sterilizer 100 initiates its operation. That is, the user can block the light with his hand, which results in a change in the current or voltage generated in the power supply 130. This change in electrical energy can be detected by the circuit element 150.

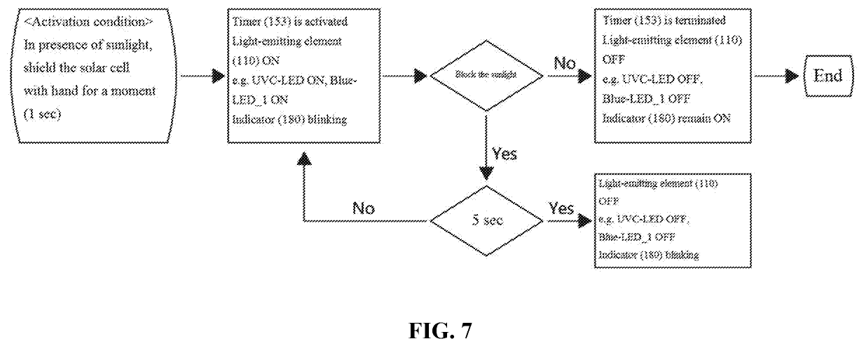

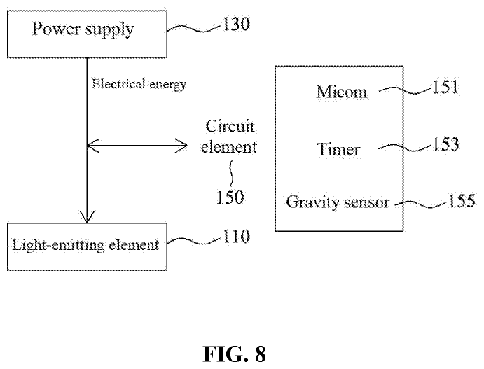

The circuit element 150 of the sterilizer 100 supplies electrical energy to the ultraviolet LED and visible LED of the light emitting element 110, as illustrated in FIG. 6D. Here, the indicator 180 of the sterilizer 100 may indicate the start of the operation to the user, by allowing the indicator 180 to blink, for example, if the indicator 180 is comprised of an LED. After the light-emitting element 110 operates for a defined period of time, its operation is terminated by a timer 153 (see FIG. 8), while the indicator 180 remains turned on (see FIG. 7).

However, if the user wants to force the sterilizer 100 to stop, as shown in FIG. 6E, he should block the light directed toward to the solar cell for at least a certain period of time.

The ultraviolet LED and visible LED of the light-emitting element 110 are then turned off, as shown in FIG. 6F. At this time, the indicator 180 of the sterilizer 100 will notify the user of this forced termination, by allowing the indicator to blink, for example, if the indicator 180 is comprised of an LED.

FIG. 7 is a flow chart describing the operations of a sterilizer according to the present disclosure.

Similar to what is shown in FIG. 6C in the situation of FIG. 6B, the user momentarily (e.g., 1 sec) shields the solar cell with his hand that serves as the power supply 130. When the generation of electrical energy by the solar cell is suspended momentarily, the solar cell undergoes electric current and/or voltage changes.

The timer 153 is activated and the light-emitting element 110 is turned on (refer back to FIG. 6D). In the flow chart, the ultraviolet LED of the light-emitting element 110 is denoted by UVC-LED and the visible LED is denoted by Blue-LED_1, for example.

The user then blocks sunlight using his hand (refer back to FIG. 6E). When the sunlight is not blocked or when the sunlight is blocked for less than 5 seconds, for example, the timer 153 is terminated and the light-emitting element 110 is turned off, i.e. UVC-LED is turned off (OFF) and Blue-LED_1 is turned off (OFF), yet the indicator 180 is continuously turned on (ON). When the sunlight is blocked for 5 seconds or longer, the light-emitting element 110 is forced to stop (e.g., UVC-LED is turned off (OFF), and Blue-LED_1 is turned off (OFF)), and the indicator 180 keeps blinking.

In other words, when the user sees that the indicator 180 is blinking with the light-emitting element 110 turned on he would know that the light-emitting element 110 is being sterilized. Moreover, when the user sees that the indicator 180 is blinking with the light-emitting element 110 turned off he would know that the light-emitting element 110 has been forced to stop. In addition, when the user sees that the indicator 180 is turned on with the light-emitting element 110 turned off he would know that the sterilization of the light-emitting element 110 for a defined period of time has been completed.

To put it in another way, if the sterilization process has been forced to stop (i.e. the light-emitting element 100 is turned off, and the indicator 180 is blinking), the user would know that a liquid (e.g., water) in the container is not sterilized and therefore it is not safe to drink. Meanwhile, if the sterilization process is completed (i.e. the light-emitting element 100 is turned off, and the indicator 180 is turned on), the user would know that it is safe to drink the liquid in the container. There are occasions when the sterilizer is forced to stop unexpectedly without the user's knowledge, such as, when a could hides the sun or when the sterilizer is put in the shade, for example. Even then, the user can easily check whether the sterilization process is completed or whether the forced termination has occurred by simply looking at the indicator 180 and the light-emitting element 110.

FIG. 8 is a schematic block diagram showing a power supply 130, a circuit element 150, and a light-emitting element 110 according to the present disclosure.

The power supply 130 provides electrical energy to the light-emitting element 110, and the circuit element 150 is arranged between the power supply 130 and the light-emitting element 110.

The circuit element 150 may include a Micom (microcomputer) 151, a timer 153, and a gravity sensor 155.

The Micom 151 can detect a change in electrical energy. The Micom 151 triggers the timer 153 based on changes in the electrical energy (e.g., voltage or current) to allow the power to be supplied from the power supply 130 to the light-emitting element 110.

The timer 153 may advantageously be used in order to prevent deterioration of the light-emitting element 110. The timer 153 may be configured to be turned on according to the pattern of changes in electrical energy stored in the Micom 151.

The gravity sensor 155 detects the posture of the sterilizer 100 (see FIG. 3). For example, if the stabilizer 100 is turned over with the light-emitting element 110 turned on, the gravity sensor 155 ensures that ultraviolet rays would not reach the eyes or body of the user.

FIG. 9 illustrates another exemplary embodiment of a sterilizer 100 according to the present disclosure.

The sterilizer 100 may be used for a container 200 with a wide opening or mouth, and for a lid 300 covering the wide opening. The lid 300 defines at least one hole 301, and a plurality of sterilizers 100 is provided in the at least one hole 301. The lid 300 can be integral with the container 200. Once the light-emitting element 110 is turned on, it starts disinfecting the container 200 as well as a liquid material inside the container 200. Each of the at least one hole 301 may have a protrusion or groove, which is intended to mesh with a corresponding groove or protrusion formed on the outer surface 173-2 of the neck portion 173 of the sterilizer 100. Although not shown, the lid 300 may include a nut about the hole 301, such that the outer surface 173-2 of the sterilizer 100 may be coupled to the nut and fixed onto the lid.

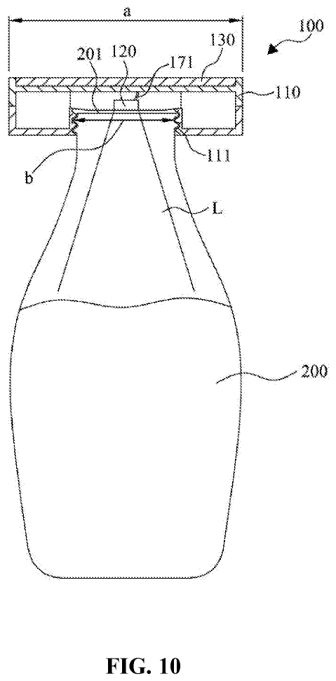

FIG. 10 is a conceptual view of a sterilizer 100 according to the present disclosure.

The sterilizer 100, which is configured to join to the container 100, includes a casing 110, a light-emitting element 120, and a solar cell 130.

The casing 110 includes a coupling portion 111 having a threaded form. The coupling portion 111 may be provided on the bottom surface of the casing 110.

The light-emitting element 120 is positioned inside the casing 110, and emits light L directed to the interior of the container 200. The light-emitting element 120 includes a first semiconductor light-emitting diode 121 (see FIG. 5), which emits ultraviolet rays. The ultraviolet rays can kill microorganisms such as bacteria. In particular, the first semiconductor light-emitting diode 121 may emit UVC rays among ultraviolet rays.

The solar cell 130 is provided in the casing 110 and may be placed toward the top surface of the casing 100. The solar cell 130 generates electrical energy from incident light. The width (a) of the solar cell 130 may be wider than the width (b) of the coupling portion 111. The width (a) and size of the solar cell 130 may determine an amount of energy that can be produced by the solar cell 130. Hence, the solar cell 130 may preferably have a width (a) greater than the width (b) of the coupling portion 111 in order to generate electrical energy required of the light-emitting element 120 to be able to operate.

The coupling portion 111 of the sterilizer 100 is coupled to the opening 201 of the container 200. The opening 201 of the container 200 is fitted into the coupling portion 111. The light-emitting element 120 emits light toward the opening 201 of the container 200. The solar cell 130 is electrically connected (as indicated by ‘171’ in the drawing) to the light-emitting element 120, such that the electrical energy (e.g., voltage) generated by the solar cell 130 would be used for the operation of the light-emitting element 120. The voltage of the solar cell 130 is preferably higher than the drive voltage of the first semiconductor light-emitting diode 121 of the light emitting element 120 by 1V to 5V. This enables the solar cell 130 to directly supply the voltage to the light-emitting element 120 without the help of a booster, resulting in a minimum power loss. Moreover, as the amount of voltage generated by the solar cell 130 can vary depending on the weather, it is necessary to have a surplus voltage, which accounts for setting up the voltage of the solar cell 1V higher than the drive voltage of the first semiconductor light-emitting diode 121. Even if the voltage of the solar cell is set higher than the drive voltage of the first semiconductor light-emitting diode 121, this would not cause a problem because the amount of current generated by a small portable solar cell 130 is usually limited anyway. However, when the voltage of the solar cell 130 is lower than the drive voltage of the first semiconductor light-emitting diode 121, a booster is needed for the first semiconductor light-emitting diode 121 to be turned on and activated. In addition, the setting of the voltage of the solar cell shall not exceed the drive voltage of the first semiconductor light-emitting diode 121 by more than 5V, in order to facilitate the manufacturing process of the solar cell 130. In general, silicon solar cells generate a voltage of about 0.5 V per cell. Therefore, in order to make an 8V solar cell, for example, 16 cells should be connected in series, complicating the overall manufacturing process. In addition, a portable sterilizer as discussed here can find a wide range of applications on condition that a smallest possible solar cell is used to fit within such a small-sized end product with the portable sterilizer. Besides, it is an exceedingly difficult process to install a large number of cells in a small area. As such, it will only increase defects and costs of the process if the solar cell 130 being manufactured would unnecessarily have a voltage 5V higher than the drive voltage of the first semiconductor light-emitting diode 121.

Usually, the container 200 is formed such that it has an opening 201 with a width smaller than the width of the lower portion of the container 200. Examples of the container 200 may include PET plastic bottles, other types of beverage bottles, and so on, which are available everywhere and have openings of similar sizes. This expands the application of the coupling portion 111 in a way of attachment to the opening of any PET plastic bottle. Moreover, these PET plastic bottles are particularly suitable for use because they do not transmit ultraviolet rays therethrough.

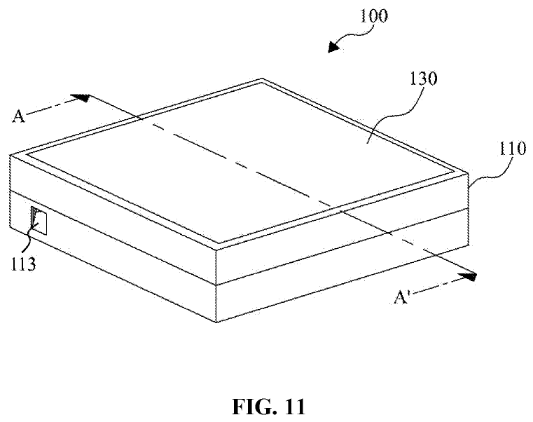

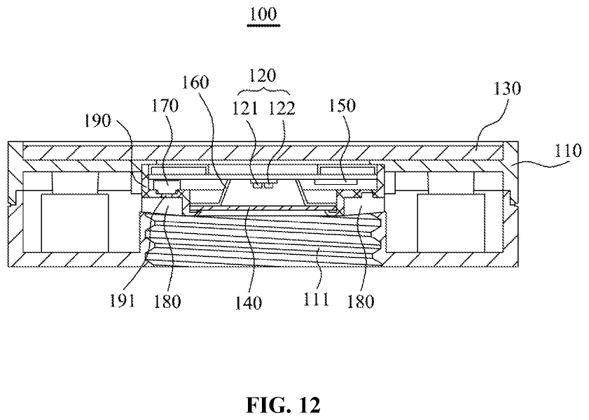

FIGS. 11 and 12 illustrate an exemplary embodiment of a sterilizer 100 according to the present disclosure.

FIG. 11 shows a perspective view of the sterilizer 100, and FIG. 12 is a cross-sectional view taken along the line AA′ of the sterilizer 100 in FIG. 11.

A light-emitting element 120 includes a second semiconductor light-emitting diode 122 which emits visible light. Because ultraviolet rays emitted from the first semiconductor light-emitting diode 121 are not visible, the user is not able to find out whether the first semiconductor light-emitting diode 121 has been turned on. To resolve this, the second light-emitting diode 122 is configured to turn on while the first semiconductor light-emitting diode 121 is being turned on, such that the user would know whether the first semiconductor light-emitting diode 121 is currently turned on. Additionally, or alternatively, the light-emitting element 120 may be configured as a semiconductor light-emitting structure (not shown) having both the first semiconductor light-emitting diode 121 and the second semiconductor light-emitting diodes 122.

The sterilizer 100 further includes a window 140, a controller 150, a reflector 160, a switch 170, an elastic member 180 and a fixing element 190.

The window 140 is arranged between the light-emitting element 120 and the coupling portion 111 and configured as a transparent part to allow light from the light-emitting element 120 can travel through it toward the container 200 (see FIG. 10). In particular, the window 140 is preferably made of a material that allows ultraviolet rays among other lights from the light-emitting element 120 to pass through the window 140 (e.g., quartz). The window 140 also serves to protect the light-emitting element 120 from the liquid contained in the container 200.

The controller 150 is electrically connected to the solar cell 130 and the light-emitting element 120 and controls the supply of electrical energy to the first semiconductor light-emitting diode 121 and the second semiconductor light-emitting diode 122, respectively. Under its control, electrical energy is supplied to the second semiconductor light-emitting diode 122 when the first semiconductor light-emitting diode 121 was turned on, but electrical energy is not supplied to the second semiconductor light-emitting diode 122 when the first semiconductor light-emitting diode 121 was turned off. The first semiconductor light-emitting diode 121 is constantly provided with electrical energy, while the second semiconductor light-emitting diode 122 is periodically provided with electrical energy. In other words, the first semiconductor light-emitting diode 121 is continuously turned on, while the second semiconductor light-emitting diode 122 may blink on and off. It is important that the first semiconductor light-emitting diode 121 remains turned on because it is the one responsible for UV sterilization. The second semiconductor light-emitting diode 122 is configured to blink on and off, instead of being constantly turned on, in order to ease difficulties of the user in noticing the on/off state of the second semiconductor light-emitting diode 122 especially when the sun is intense or any incident light from outside is too bright. With the second semiconductor light-emitting diode 122 flickering, the user can easily see whether the second semiconductor light-emitting diode 122 is being turned on. Additionally, or alternatively, the controller 150 may have the second semiconductor light-emitting diode 122 get the supply of pulsed electrical energy, for example, such that part of the electrical energy required to turn on the second semiconductor light-emitting diode 122 can be saved. Additionally, or alternatively, for better noticeability to the user, the second semiconductor light-emitting diode 122 may be configured to shed light of more intense brightness when it blinks, by increasing the magnitude of electrical energy being supplied to the second semiconductor light-emitting diode 122. As mentioned previously, this blinking mode of the second semiconductor light-emitting diode 122 is designed to save the electrical energy generated by the solar cell 130. The larger the solar cell 130 is, the greater electrical energy can be supplied. However, with the solar cell 130 having a limited size, it would be more desirable to save the electrical energy generated therefrom.

The reflector 160 encircles the light-emitting element 120 and reflects light coming out on the sides of the light-emitting element 120 to guide the light toward the opening 201 (see FIG. 3) of the container 200. The reflector 160 preferably has a smaller width than the width of the opening 201 of the container 200. In this way, the light for use in sterilization of the liquid in the container 200 would not collide with the surfaces of the opening 201 on its way toward the liquid. As the opening 201 of the container 200 has a smaller width that the width of the lower portion of the container 200, it becomes important to ensure that the light from the first semiconductor light-emitting diode 121 is well delivered to the lower part of the container 200. It is understood that light from a semiconductor light emitting element has a wide emission angle of 120 degree or more, meaning that a large portion of the light would be lost while traveling down to the lower part of the container 200. Unlike those in the visible light region, UVC rays are mostly absorbed when they collide with a surface made from plastics. As such, to benefit from high-level sterilization, it is important to ensure that much of the light from the first semiconductor light-emitting diode 121 would be delivered to the lower portion of the container 200, without causing light loss due to possible collisions with the container 200. This can be accomplished by arranging the reflector 160 around the first semiconductor light-emitting diode 121 and producing the reflector 160 with a material having high UVC reflectance. For example, the reflector 160 may be made of aluminum, Teflon, or the like. Preferably, the reflector 160 is made of aluminum, which is easier to mold and inexpensive.

The switch 170 is positioned between the light-emitting element 120 and the solar cell 130 and serves to electrically connect them (see 171 in FIG. 10). The light-emitting element 120 and the solar cell 130 are electrically connected to or electrically disconnected from each other as the switch 170 is either in ON or OFF position. When the switch 170 is turned on, the light-emitting element 120 and the solar cell 130 are electrically connected 171, allowing the electrical energy generated by the solar cell 130 to be supplied to the light-emitting element 120 to turn it on. Meanwhile, when the switch 170 is turned off, the light-emitting element 120 and the solar cell 130 are electrically disconnected from each other, cutting off the supply of electrical energy from the solar cell 130 to the light-emitting element 120. As a result, the light-emitting element 120 is turned off.

The elastic member 180 is provided between the coupling part 111 and the container 200. It is formed along the rim of the opening 201 of the container 200. The elastic member 180 together with the window 140 can seal the casing 110. More specifically, the elastic member 180 and the window 140 serve to prevent inflow of the liquid in the container 200 into the casing 110. The elastic member 180 may be made of an elastic material such as silicone or rubber, for example.

The fixing element 190 is positioned to come in contact with the elastic member 180 and the window 140, so as to fix both. The fixing element 190 has a hole 191, allowing the elastic member 180 and the switch 170 to contact each other therethrough.

The first semiconductor light-emitting diode 121 of the light emitting element 120 emits UVC rays. It is understood that direct exposure to UVC rays can damage the body and cause eye problems. Because of that, the light-emitting element 120 is allowed to operate only after the container 200 is properly joined to the coupling portion 111. Once the container 200 is placed on the sterilizer 100 along the coupling portion 111, the elastic member 180 is pressed by the opening 201 of the container 200 and as a result, the elastic member 180 presses the switch 170 positioned in contact with the elastic member 180, such that the switch 170 turns from off to on. On the other hand, when the container 200 is released and the elastic member 180 and the opening 201 of the container 200 are thus separated, the elastic member 180 is no longer under the pressing force of the opening 201 of the container 200, such that the switch 170 turns from on to off. The fixing element 190, which is arranged between the elastic member 180 and the switch 170, has the hole 191, allowing the elastic member 180 and the switch 170 to come in contact with each other through the hole 191, as mentioned previously. For example, a portion of the elastic member 180 may extend through the hole 191 to contact the switch 170, or vice versa, i.e. a portion of the switch 170 may extend through the hole 191 to contact the elastic member 180. After all, even if the solar cell 130 receives sunlight and generates electrical energy, the operation of the light-emitting element 120 is triggered only after the container 200 and the coupling portion 111 are properly joined together, which ensures overall safety of the sterilizer.

In addition, the casing 110 further includes a connection part 113 (see FIG. 11) which is configured to penetrate a part of the casing 110. An elongated item such as a string, a strap, or even a thread can pass through this connection part 113, in order to provide the sterilizer 100 with portability and convenience of the usage. In one example, the sterilizer 100 can be carried around using a strap as a lanyard for example or can be configured as a holder attached to a bag or any other portable item. If the casing 110 is a cuboid for example, the connecting part 113 can be formed at one corner of the casing 110 and a strap may be attached to the connecting part 113, functioning similarly to a lanyard. When worn around the neck, the casing 110 would be tilted over (e.g., the rectangle shape being slanted into a rhombus), which ensures safer and more stable usage on the user's end.

The casing 110 may further include a timer (not shown). The timer can be electrically connected between the light-emitting element 120 and the solar cell 130. After voltage is supplied from the solar cell 130 to the light-emitting element 120 for a defined period of time, the timer is activated to cut off the voltage supply. That is, after the switch 170 is turned on, voltage is supplied to the light-emitting element 120 for a certain period of time until it is cut off by the timer.

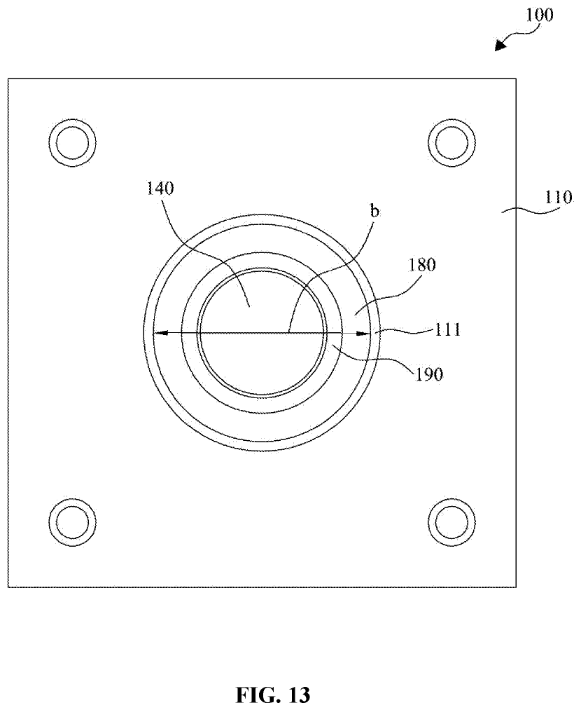

FIG. 13 illustrates another exemplary embodiment of a sterilizer 100 according to the present disclosure.

FIG. 13 shows a rear view of the sterilizer 100. A casing 110 has a coupling portion 111 on the bottom surface of the casing 110. The coupling portion 111 is formed on the inside lateral surface of the casing 110. With this configuration, the coupling portion 111 as well as the light-emitting element 120 (FIG. 12) are positioned inside the casing 110, and nothing is stretched outwardly from the casing 110. Therefore, both the coupling portion 111 and the light-emitting element 120 can be protected from external impact, when the user carries the sterilizer 100.

When seen from the bottom of the casing 110, the coupling portion 111, the elastic member 180, and the window 140 are all exposed. The elastic member 180 is configured to surround the window 140, and a fixing element 190 is provided between the elastic member 180 and the window 140, so as to fix both.

Set out below are a series of clauses that disclose features of further exemplary embodiments of the present disclosure, which may be claimed.

(1) A sterilizer for sterilizing a container with ultraviolet rays comprises: a light-emitting element including at least one semiconductor light-emitting diode that emits ultraviolet rays directed to the container; a power supply for supplying electrical energy to the light-emitting element; a casing having a main body in which the power supply is provided, and a neck portion lying under the main body, the neck portion being configured to join to the container; and a circuit element for detecting a change in electrical energy of the power supply to control the light-emitting element.

(2) There is also provided, the sterilizer of clause (1) wherein: the light-emitting element is positioned inside the neck portion, and protrusions are formed within the neck portion, surrounding the light-emitting element.

(3) There is also provided, the sterilizer of clause (1) wherein: the circuit element includes a gravity sensor for detecting the posture of the sterilizer, the circuit element being configured to turn off the light-emitting element if the casing is turned over.

(4) There is also provided, the sterilizer of clause (1) wherein: the main body has a width greater than the width of the neck portion, which is extended outwardly from the lower part of the main body.

(5) There is also provided, the sterilizer of clause (1) wherein: the circuit element detects electrical energy from the power supply, the circuit element being adapted to allow or cut off electrical connection between the power supply and the light-emitting element based on changes in the electrical energy.

(6) There is also provided, the sterilizer of clause (1) further comprising: an indicator for indicating a forced stop or forced termination of sterilization during a sterilization process.

(7) There is also provided, the sterilizer of clause (1) wherein: the neck portion has an outer surface and an inner surface, the outer surface and the inner surface having at least one of grooves or protrusions formed thereon.

(8) There is also provided, the sterilizer of clause (1) wherein: the neck portion has an outer surface and an inner surface, each of the outer surface and the inner surface being configured to join to the container.

(9) A sterilizer for sterilizing a container with ultraviolet rays comprises: a light-emitting element including at least one semiconductor light-emitting diode that emits ultraviolet rays directed to the container; a solar cell for supplying electrical energy to the light-emitting element; and a casing having a main body in which the solar cell is provided, and a neck portion extending downward on opposite sides of the solar cell, the neck portion having an outer surface and an inner surface, wherein the container joins to the outer surface and the inner surface of the neck portion.

(10) There is also provided, the sterilizer of clause (9) wherein: the outer surface and the inner surface of the neck portion have at least one of grooves or protrusions formed thereon.

(11) A sterilizer coupled to a container comprises: a casing including a coupling portion to join to the container; a light-emitting element for emitting light directed to the interior of the container, the light-emitting element including a first semiconductor light-emitting diode for emitting ultraviolet rays; and a solar cell provided in the casing, the solar cell being electrically connected with the light-emitting element to supply electrical energy to the light-emitting element.

(12) There is also provided, the sterilizer of clause (11) wherein: the coupling portion is recessed inward from the bottom surface of the casing.

(13) There is also provided, the sterilizer of clause (11) wherein: the coupling portion is formed on the inside lateral surface of the casing.

(14) There is also provided, the sterilizer of clause (11) further comprising: an elastic member provided in the casing and arranged to be pressed by the container.

(15) There is also provided, the sterilizer of clause (11) further comprising: a switch configured to allow or cut off electrical connection between the light-emitting element and the solar cell.

(16) There is also provided, the sterilizer of clause (11) further comprising: an elastic member provided in the casing and arranged to be pressed by the container; and a switch configured to allow or cut off electrical connection between the light-emitting element and the solar cell, wherein the switch is activated as the elastic member pressed by the container pushes the switch.

(17) There is also provided, the sterilizer of clause (11) further comprising: an elastic member provided in the casing and arranged to be pressed by the container; and a switch configured to allow or cut off electrical connection between the light-emitting element and the solar cell, wherein the switch and the elastic member are arranged to come in contact with each other.

(18) There is also provided, the sterilizer of clause (11) wherein: the switch remains turned on while the coupling portion and the container are being joined to each other.

(19) There is also provided, the sterilizer of clause (11) further comprising: a window provided in a direction where light from the light-emitting element travels.

(20) There is also provided, the sterilizer of clause (11) further comprising: a reflective wall encircling the light-emitting element, the reflective wall being configured to converge light from the light-emitting element and to direct the light to the interior of the container.

With an exemplary sterilizer according to the present disclosure, the container can be fixed to both the outside and the inside of the neck portion.

With another exemplary sterilizer according to the present disclosure, the container can be sterilized without using an external power supply.

Another exemplary sterilizer according to present disclosure features portability and convenience of the usage.

With another exemplary sterilizer according to present disclosure, the container can be sterilized without requiring electricity.

What is claimed is:

1. A sterilizer coupled to a container comprising:
a casing including a coupling portion to join to the container;
a light-emitting element for emitting light directed to the interior of the container, the light-emitting element including a first semiconductor light-emitting diode for emitting ultraviolet rays; and
a solar cell provided in the casing, the solar cell being electrically connected with the light-emitting element to supply electrical energy to the light-emitting element.

2. The sterilizer according to claim 1, wherein the coupling portion is recessed inward from the bottom surface of the casing.

3. The sterilizer according to claim 2, wherein the coupling portion is formed on the inside lateral surface of the casing.

4. The sterilizer according to claim 3, further comprising:
an elastic member provided in the casing and arranged to be pressed by the container.

5. The sterilizer according to claim 4, further comprising:
a switch configured to allow or cut off electrical connection between the light-emitting element and the solar cell.

6. The sterilizer according to claim 1, further comprising:
an elastic member provided in the casing and arranged to be pressed by the container; and
a switch configured to allow or cut off electrical connection between the light-emitting element and the solar cell, wherein the switch is activated as the elastic member pressed by the container pushes the switch.

7. The sterilizer according to claim 1, further comprising:
an elastic member provided in the casing and arranged to be pressed by the container; and
a switch configured to allow or cut off electrical connection between the light-emitting element and the solar cell, wherein the switch and the elastic member are arranged to come in contact with each other.

8. The sterilizer according to claim 7, wherein the switch remains turned on while the coupling portion and the container are being joined to each other.

9. The sterilizer according to claim 1, further comprising:
a window provided in a direction where light from the light-emitting element travels.

10. The sterilizer according to claim 1, further comprising:
a reflective wall encircling the light-emitting element, the reflective wall being configured to converge light from the light-emitting element and to direct the light to the interior of the container.
